(19) United States
(12) Patent Application Publication
(10) Pub. No.: US 2013/0102062 A1
(43) Pub. Date: Apr. 25, 2013

(54) MICROCHIP FOR NUCLEIC ACID AMPLIFICATION REACTION AND METHOD OF PRODUCING THE SAME

(71) Applicant: Sony Corporation, Tokyo (JP)

(72) Inventors: Kensuke Kojima, Kanagawa (JP); Masaki Sato, Tokyo (JP); Masahiro Matsumoto, Kanagawa (JP)

(73) Assignee: SONY CORPORATION, Tokyo (JP)

(21) Appl. No.: 13/655,156

(22) Filed: Oct. 18, 2012

(30) Foreign Application Priority Data
Oct. 25, 2011 (JP) 2011-233681

(51) Int. Cl. C12M 1/40

(52) U.S. Cl. 435/287.1

(57) ABSTRACT

Provided is a microchip for a nucleic acid amplification reaction, including a well configured to function as a reaction site of the nucleic acid amplification reaction, and the well has a center portion and a marginal portion, a substance anchored in a form that the substance is eccentrically-located less at the center portion and much at the marginal portion of the well, in which the substance is at least a part of a substance for the nucleic and amplification reaction.

### CROSS REFERENCES TO RELATED APPLICATIONS

The present application claims priority to Japanese Priority Patent Application JP 2011-233681 filed in the Japan Patent Office on Oct. 25, 2011, the entire content of which is hereby incorporated by reference.

### BACKGROUND

The present disclosure relates to a microchip for a nucleic acid amplification reaction and a method of producing the same, more particularly to a microchip for a nucleic acid amplification reaction in which a reagent containing one or more components for the reaction is anchored in a certain form within a well configured to function as a reaction site of the nucleic acid amplification reaction, or the like.

In recent years, by applying a microfabrication technique in the semiconductor industry, microchips having wells and flow channels for performing chemical and biological analyses formed on a substrate made of silicon or glass have been developed. These microchips have begun to be used, for example, for an electrochemical detector of liquid chromatography or a small-sized electrochemical sensor in a practical medical field.

An analysis system using such a microchip is referred to as u-TAS (micro-Total-Analysis System), Lab-on-chip, biochip or the like, and attracts attention as a technology that can speed up, increase efficiency of, and integrate the chemical and biological analyses, or decrease a size of an analysis apparatus. Since the u-TAS can analyze a small amount of samples and the microchips can be disposable (single-use), it is expected to apply it to the biological analysis that handles, specifically, a trace amount of precious samples or many test bodies.

An example of the application of the μ-TAS is an optical detector which introduces substances into a plurality of areas provided in microchips, and chemically detects the substances. An example of the optical detector is a reaction apparatus (for example, a real time PCR apparatus) that causes a reaction of a plurality of substances such as a nucleic acid amplification reaction to proceed in wells of microchips, and optically detects substances produced.

In related art, a microchip type nucleic acid amplification apparatus adopts a method to perform the reaction, by mixing all reagents for a nucleic acid amplification reaction and template DNAs in advance, and introducing the mixed liquid into a plurality of wells provided in microchips. In this method, however, it takes a certain period of time to introduce the mixed liquid into the wells. There is a problem that the reaction proceeds in the mixed liquid during the time period, non-specific amplification is facilitated, and quantitative performance decreases.

As to the above-mentioned problem, Japanese Unexamined Patent Application Publication No. 2007-43998 discloses, for example, a microchip holding a part of a solid-state reagent for a nucleic acid amplification reaction in a flow channel. There is no description about a form of the solid-state reagent and a detailed position within the microchip disclosed in Japanese Unexamined Patent Application Publication No. 2007-43998.

### SUMMARY

Thus, it is desired to provide a microchip for a nucleic acid amplification reaction and a method of producing the same simply with high accuracy.

The present inventors have focused on and studied about an anchored form of a reagent for a nucleic acid amplification reaction within a microchip. As a result, they have found that the reagent is anchored in a form that the reagent is eccentrically-located less at a center portion and much at a marginal portion of a well, by applying a hydrophilic treatment to the inner surface of the well of the microchip, to which the reagent is anchored, dropping a solution containing a substance for a nucleic acid amplification reaction into the well and drying the solution. The reagent anchored within the well has a large surface area with a certain uniform form, is easily dissolved once the nucleic acid amplification reaction is started, and has decreased variations between the wells.

According to an embodiment of the present disclosure, there is provided a microchip for a nucleic acid amplification reaction including a well configured to function as a reaction site of the nucleic acid amplification reaction, in which at least a part of a substance for the nucleic acid amplification reaction is anchored in a form that the substance is eccentrically-located less at a center portion and much at a marginal portion of the well.

The microchip for the nucleic acid amplification reaction has desirably the well having a hydrophilic surface.

Desirably, in the microchip for the nucleic acid amplification reaction, the substance has a cone-shaped form that reduces a height from the marginal portion to the center portion of the well.

More desirably, in the microchip for the nucleic acid amplification reaction, the substance is placed circularly around the area excluding the center portion of the well.

According to an embodiment of the present disclosure, there is provided a method of producing a microchip for a nucleic acid amplification reaction including applying a hydrophilic treatment to a surface of a substrate layer, on which a well configured to function as a reaction site of the nucleic acid amplification reaction is formed, dropping at least a part of a substance for the nucleic acid amplification reaction into the well, and drying and anchoring the substance within the well.

In the method of producing the microchip for the nucleic acid amplification, a hydrophilic treatment on the surface on which the well is formed includes desirably exposing the surface to plasma.

In the method of producing the microchip for the nucleic acid amplification reaction, the anchoring the substance within the well includes desirably freeze drying the substance dropped into the well.

According to an embodiment of the present disclosure, there is provided the microchip for the nucleic acid amplification reaction simply with high accuracy.

These and other objects, features and advantages of the present disclosure will become more apparent in light of the following detailed description of best mode embodiments thereof, as illustrated in the accompanying drawings.

Additional features and advantages are described herein, and will be apparent from the following Detailed Description and the figures.

### BRIEF DESCRIPTION OF THE FIGURES

FIG. 1 is a schematic top view of a microchip for a nucleic acid amplification reaction according to an embodiment of the present disclosure;

FIGS. 2A and 2B are schematic views each illustrating a form of a reagent anchored within wells;

FIGS. 3A and 3B are schematic partial sectional views of the microchip for the nucleic acid amplification reaction according to the embodiment of the present disclosure (FIG. 1: P-P′ section);

FIG. 4 is a flowchart illustrating a method of producing the microchip for the nucleic acid amplification reaction according to the embodiment of the present disclosure;

FIGS. 5A through 5C are photographs substituted for drawings each showing a form of a reagent anchored within wells in a microchip for a nucleic acid amplification reaction in Example; and

FIGS. 6A through 6C are photographs substituted for drawings each showing a state of the dissolved reagent anchored within the wells in the microchip for the nucleic acid amplification reaction in Example.

### DETAILED DESCRIPTION

Hereinafter, favorable embodiments for carrying out the present disclosure will be described. It should be noted that the embodiments described below illustrate only examples of typical embodiments of the present disclosure, and the scope of the present disclosure is not narrowly interpreted by the embodiments. The embodiments will be described in the following order.

1. Microchip for nucleic acid amplification reaction according to embodiment of present disclosure
(1-1) Configuration of microchip for nucleic acid amplification reaction
(1-2) Reagent for nucleic acid amplification reaction
(1-3) Branched flow channel and communicating part of well
2. Method of producing microchip for nucleic acid amplification reaction according to embodiment of present disclosure
(2-1) Formation of substrate layer
(2-2) Hydrophilic treatment within well
(2-3) Dropping of reagent into well
(2-4) Anchoring of reagent within well
(2-5) Bonding of substrate layers

1. Microchip for Nucleic Acid Amplification Reaction According to Embodiment of Present Application

A microchip for a nucleic acid amplification reaction according to an embodiment of the present disclosure (hereinafter referred to as a “microchip”) will be described. “The nucleic acid amplification reaction” that uses the microchip according to the embodiment of the present disclosure includes a PCR (Polymerase Chain Reaction) method in the past involving a temperature cycle and various isothermal amplification methods involving no temperature cycle. Examples of the isothermal amplification methods include a LAMP (Loop-Mediated Isothermal Amplification) method, a SMAP (Smart Amplification Process) method, a NASBA (Nucleic Acid Sequence-Based Amplification) method, an ICAN (Isothermal and Chimeric primer-initiated Amplification of Nucleic acids) Method™, a TRC (Transcription-Reverse transcription Concerted) method, an SDA (Strand Displacement Amplification) method, a TMA (Transcription-Mediated Amplification) method, an RCA (Rolling Circle Amplification) method and the like. “The nucleic acid amplification method” involves a wide variety of other poikilothermal or isothermal nucleic acid amplification reactions for amplifying nucleic acids. These nucleic acid amplification reactions involve a reaction for quantifying the amplified nucleic acid such as a real time PCR method.

(1-1) Configuration of Microchip for Nucleic Acid Amplification Reaction

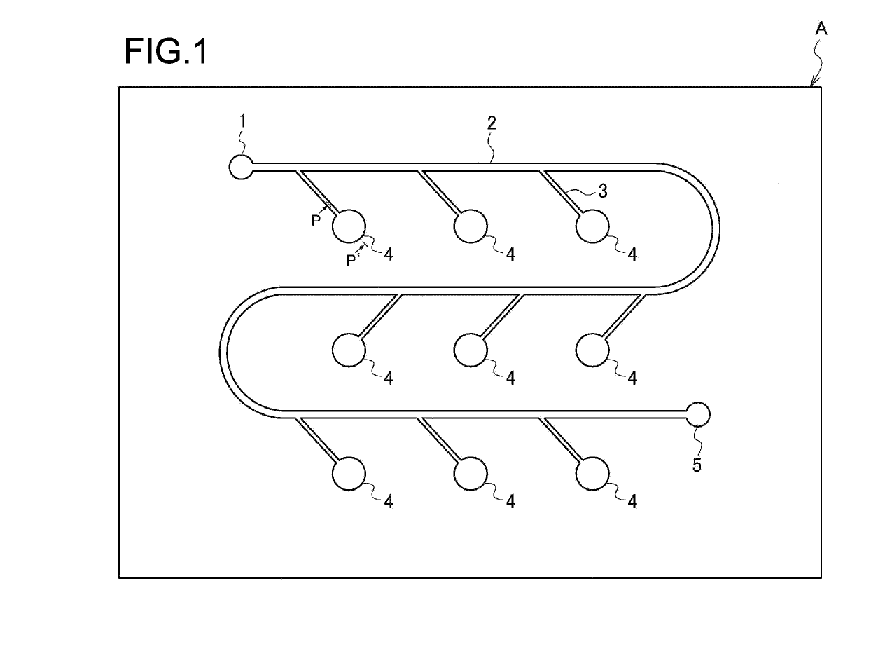

FIG. 1 is a schematic top view of a microchip for a nucleic acid amplification reaction according to an embodiment of the present disclosure. A microchip A includes a feed port 1 into which a sample solution is fed externally, a well 4 configured to function as a reaction site of the nucleic acid amplification reaction, a main flow channel 2 communicating with the feed port 1 at one end and with an outlet 5 at other end, and a branched flow channel 3 branched from the main flow channel 2 and communicating with the well 4. Herein, the sample solution refers to a solution containing a nucleic acid such as DNA, RNA, etc., which is a template nucleic acid to be amplified in the nucleic acid amplification reaction.

The microchip A includes a plurality of substrate layers. The substrate layers can be made with glass and a variety of plastics (polypropylene, polycarbonate, cyclo olefin polymer, polydimethylsiloxane etc.). Desirably, the material of the substrate layer has light permeability, less autofluorescence and little wavelength dispersion, which causes less optical errors, when nucleic acid strands amplified within the well 4 are detected or quantified optically. The microchip A includes a plurality of substrate layers, and their numbers are not limited. Also, the microchip A can be made by bonding a plurality of materials, e.g., by bonding a glass substrate layer with a plastic substrate layer. Even when any materials are used as the substrate layers, the inner surface of the well 4 disposed on the substrate layer is desirably hydrophilic. The hydrophilic treatment of the inner surface of the well 4 will be described in (2-2).

Although FIG. 1 illustrates nine wells 4 disposed on the microchip A, any numbers of the wells 4 may be disposed on one microchip, and a form of the well 4 may not be limited to a cylindrical shape. Also, the main flow channel 2 and the branched flow channel 3 formed on the microchip are not limited to the embodiment shown in FIG. 1. One microchip may include a plurality of feed ports 1 and a plurality of main flow channels 2. The outlet 5 for discharging the sample solution fed into the microchip A from the feed port 1 to outside may not be provided. The sample solution may not be discharged to outside.

(1-2) Reagent for Nucleic Acid Amplification Reaction

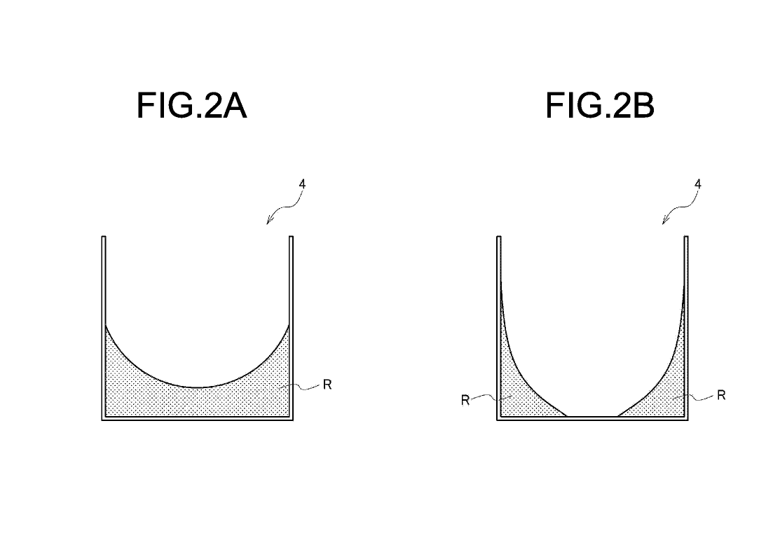

FIG. 2 are schematic views each illustrating a form of a reagent for the nucleic acid amplification reaction (hereinafter referred to as a “reagent”) within the well 4 of the microchip A according to the embodiment of the present disclosure. A reagent R is anchored in the inner surface of the well 4 in the form that the reagent is eccentrically-located less at a center portion and much at a marginal portion of the well 4. For example, the eccentrically-located form is a cone-shaped form that reduces a height from the marginal portion to the center portion of the well 4, as shown in FIG. 2A. FIG. 2B shows an alternative embodiment of the embodiment according to the present disclosure. When the reagent R in a cone-shaped form reduces the bulk from the marginal portion to the center portion of the well 4 and the bulk is lost before arriving at the center portion of the well 4, the reagent R is placed circularly around the area excluding the center portion of the well 4 as shown in FIG. 2B.

The reagent R anchored within the well 4 contains at least a part of a substance for providing amplified nucleic acid strands in the nucleic acid amplification reaction. Specific examples include an oligonucleotide primer (hereinafter referred to as a “primer”) complementary to at least a part of a base sequence such as DNA and RNA to be amplified, nucleic acid monomers (dNTPs), enzymes, components contained in a reaction buffer solution and the like. As the substance for detection of the amplified nucleic strands, the reagent R can contain a probe having a label such as a fluorescent label for detection of the amplified nucleic acid and a detection reagent for intercalating double-stranded nucleic acids, although these are not expected to be contained directly for the nucleic acid amplification reaction. A component contained in the reagent R anchored within the well 4 is one or more of the substances for the nucleic acid amplification reaction and the detection. When a plurality of components are contained in the reagent R, the reagent R can have a single layer including the plurality of components uniformly mixed. Or, the reagent R may have a laminate structure having the plurality of components laminated in order within the well 4.

Kinds of the substances contained in one layer of the reagent R laminated within the well 4 are not especially limited. The numbers of the layers of the reagent R in one well 4 are also not limited. The order in which the substances are laminated within the well 4 can be arbitrary. For example, there may be a four-layered structure of “the primer, dNTPs, enzymes, the components contained in the reaction buffer” or a three-layered structure of “the components contained in the reaction buffer, enzymes, dNTPs and the primer” with a plurality of substances for the nucleic acid amplification reaction being mixed in one layer in advance. Specifically, when the primer layer is laminated as a bottom layer and the layer containing other reagents is laminated as a top layer within the well 4, non-specific amplification of the nucleic acids is favorably prevented upon the start of the nucleic acid amplification reaction, as the nucleic acid amplification reaction is started only after the bottom layer containing the primer is dissolved. When one microchip A has a plurality of wells 4, the kinds and the numbers of the lamination layers of the reagent R may not be the same between the wells 4. For example, in the nine wells 4 in the microchip A shown in FIG. 1, the reagents R having different lamination layers including different substances may be anchored: the reagent R including only one substance for the nucleic acid amplification reaction, the reagent R having one layer including a plurality of substances for the reaction, the reagent R having lamination layers including a plurality of substances for the reaction in order and the like.

The reagent R has a layered structure including a plurality of substances for the nucleic acid amplification reaction, so that the substances for a variety of reactions can be held within the microchip A in a separated state different from the case that a mono layer is used, and the kinds of the substances added to the sample solution containing the nucleic acids to be amplified can be decreased when the nucleic acid amplification reaction is started. As a result, the microchip A according to the embodiment of the present disclosure can perform an analysis more conveniently. Also, mixing of the substances for an amplification reaction is prevented until the nucleic acid amplification reaction is started, so that the non-specific amplification of a primer dimer or the like can be inhibited, whereby the analysis with high accuracy can be performed using the microchip A.

(1-3) Branched Flow Channel and Communicating Part of Well

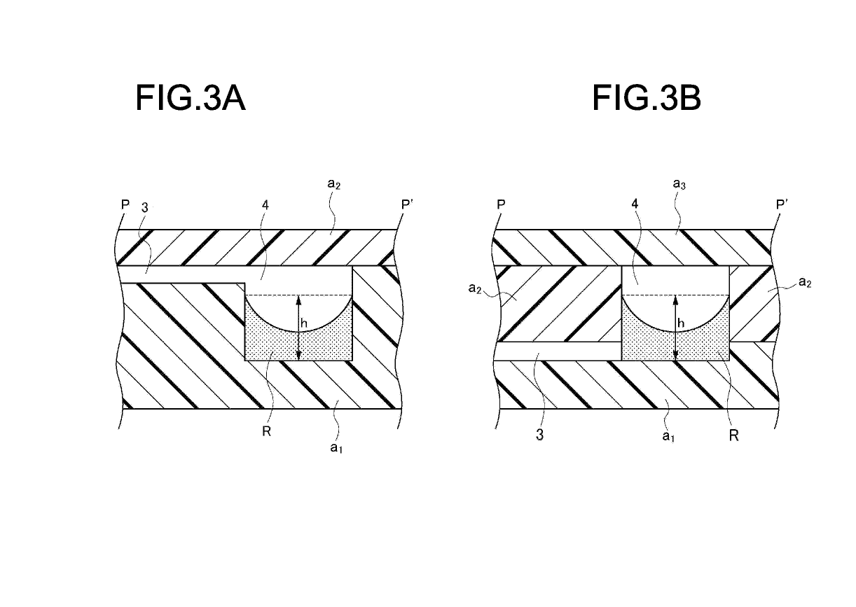

In the microchip A, the sample solution fed from the feed port 1 flows through the main flow channel 2, is branched at the branched flow channel 3 and reaches the well 4. The branched flow path 3 can be communicated with the well 4 at any side surface thereof regardless of the volume of the reagent R anchored within the well 4. In the microchip A according to the embodiment of the present disclosure, a communicating part of the branched flow channel 3 and the well 4 is desirably at a position higher than the thickest part of the substance for the nucleic acid amplification reaction anchored within the well 4. FIG. 3A is a schematic partial sectional view corresponding to P-P′ section in FIG. 1, and FIG. 3B shows the alternative embodiment. FIGS. 3A and 3B illustrate the form of the communicating part.

In the partial P-P′ section shown in FIG. 3A, the microchip A includes two substrate layers: a substrate layer a₁ on which the branched flow channel 3 and the well 4 are formed is bonded to a substrate layer a_{z}. The communicating part of the branched flow channel 3 and the well 4 is disposed at side surface of the well 4 at a position higher than the thickest height h of the reagent R anchored within the well 4 such that the reagent R does not fill the communicating part. Therefore, the sample solution flows through the branched flow channel 3, is fed into the well 4 from the communicating part, and is contacted with the surface of the reagent R anchored. The reagent R is eccentrically-located less at a center portion and much at a marginal portion of the well 4, is a cone shape or circular, has a larger contact area with the sample solution, and is dissolved promptly once the sample solution is fed. In FIG. 3, the form of the reagent R is similar to that in FIG. 2A, but may be the form shown in FIG. 2B, or any form as long as it is eccentrically-located less at a center portion and much at a marginal portion of the inner surface of the well 4.

In the partial P-P′ section shown in FIG. 3B, the microchip A includes three substrate layers: a substrate layer a_{z} having a hollow for the well 4, a substrate layer a₁, on which the bottom of the branched flow channel 3 is formed bonded to one surface of the substrate layer a_{z}, and a substrate layer a₃ bonded to other surface thereof. The communicating part of the branched flow channel 3 and the well 4 is disposed at side surface of the well 4 at a position lower than the thickest height h of the reagent R anchored within the well 4 such that the reagent R fills the communicating part. Therefore, the sample solution flows through the branched flow channel 3, and is fed into the well 4 while dissolving the reagent R. The sample solution reaches the communicating part, penetrates through the reagent R, reaches the center portion of the well 4, and is contacted with the surface of the reagent R similar to as in FIG. 3A. In FIG. 3B, a check valve is desirably disposed at the communicating part of the branched flow channel 3 and the well 4 in order to prevent a counter flow of a reaction liquid from the well 4 to the branched flow channel 3 after the sample solution is fed, as the communicating part is disposed lower than the height h of the thickest part of the reagent R.

In the microchip A having the above-described configuration according to the embodiment of the present disclosure, the nucleic acid amplification reaction can be started by feeding only remaining substances for the nucleic acid amplification reaction not anchored within the well 4 and the sample solution containing a template DNA or RNA to be amplified. In the microchip according to the embodiment of the present disclosure, as the reagent R anchored within the well 4 is eccentrically-located less at a center portion and much at a marginal portion of the well 4 and is a cone shape or circular, variations in mixing of the reagent R and the like between the wells 4 can be avoided when the reaction is started. In addition, when the reagent R is thin in the center portion of the well 4 or when the reagent R anchored within the well 4 is absent in the center portion, the reagent R has a large surface area and is easily dissolved upon mixing with the sample solution, and it prevents the reagent R from not dissolving and remaining on the center portion of the well 4. Accordingly, it can avoid detecting extremely high signal intensity on the center portion of the well 4 because of the reagent R not dissolved and remained, for example, when nucleic acids are optically detected.

2. Method of Producing Microchip for Nucleic Acid Amplification Reaction According to Embodiment of Present Application

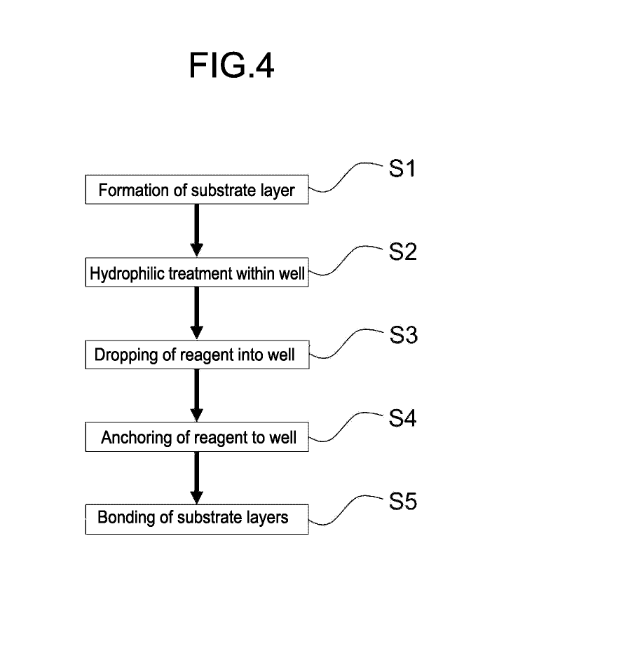

A method of producing the microchip for the nucleic acid amplification reaction according to the embodiment of the present disclosure (hereinafter referred to as a “microchip”) will be described based on a flowchart shown in FIG. 4.

(2-1) Formation of Substrate Layer

In FIG. 4, a symbol S1 represents a formation of a substrate layer. In S1, on the substrate layer, the feed port 1, the main flow channel 2, the branched flow channel 3, the well 4 and the outlet 5, all of which are shown in FIG. 1, are formed. Upon the formation, a glass substrate layer can be wet etched or dry etched, and a plastic substrate layer can be nanoimprinted, injection molded or machined. Each component such as the well 4 can be formed only on the substrate layer a₁ as shown in FIG. 3 or on a plurality of substrate layers as shown in FIG. 3B, which may be selected depending on the positions in the microchip of each component such as the main flow channel 2, the branched flow channel 3 and the well 4.

(2-2) Hydrophilic Treatment within Well

In FIG. 4, a symbol S2 represents a hydrophilic treatment of the inner surface of the well 4. In S2, the inner surface of the well 4 formed on the substrate layer in the formation S1 as described above is subjected to the hydrophilic treatment. The hydrophilic treatment includes application of a hydrophilic resin, a photocatalytic surface treatment, an inorganic-based coating such as an alkali silicate coating, an etching treatment, a plasma treatment and the like. Alternatively, an emboss (concavo-convex) form may be made by a stamper on the inner surface of the well 4 when the substrate layer is formed as the hydrophilic treatment. In the microchip A according to the embodiment of the present disclosure, a desirable hydrophilic treatment is to expose the inner surface of the well 4 to plasma.

(2-3) Dropping of Reagent into Well

In FIG. 4, a symbol S3 represents a dropping of the reagent R containing at least a part of the substance for the nucleic acid amplification reaction into the well 4 of the microchip A. In S3, the reagent R containing the substance for the amplification reaction in the form of a liquid or a gel is dropped into the well 4 that is subjected to the hydrophilic treatment in S2 as described above. Droplets of the reagent R dropped into the well 4 are eccentrically-located less at a center portion and much at a marginal portion within the well 4 by the hydrophilic treatment of the surface of the well 4. The form of the reagent R located eccentrically within the well 4 may be, for example, a cone shape as shown in FIG. 2A, or circular as shown in FIG. 2B, depending on the degree of the hydrophilicity on the inner surface of the well 4, the amount and properties of the reagent R and the like.

During the dropping of the reagent R into the well 4, the components contained in the reagent R may be subjected to any predetermined treatment(s) as appropriate, before the reagent R is dropped. For example, when the reagent R containing the primer is dropped, the reagent R is treated at a temperature of about 95° C. in advance to modify the primer into a single-strand primer, so that the primer dimer can be less produced and the non-specific amplification of nucleic acids can be decreased upon the nucleic acid amplification reaction.

In FIG. 4, the dropping S3 of the reagent R into the well 4 is subsequent to the formation S1 of the substrate layer and the hydrophilic treatment S2 on the inner surface of the well 4. In the method of producing the microchip A according to the embodiment of the present application, by preparing the substrate layer on which the well 4 and the like are formed, which is subjected to the hydrophilic treatment, the dropping S3 into the well 4 may be started.

(2-4) Anchoring of Reagent within Well

In FIG. 4, a symbol S4 represents a drying of the reagent R within the well 4 and an anchoring the reagent R to the inner surface of the well 4. The reagent R dropped into the well 4 in the dropping S3 of the reagent R keeps the form that the reagent R is eccentrically-located less at a center portion and much at a marginal portion of the well 4 by the hydrophilic treatment of the inner surface of the well 4. In S4, the reagent R is dried while the form of the reagent R is kept, and is anchored within the well 4. Freeze drying is desirably used. Before the freeze drying, the reagent R may be cooled for sufficient freezing in advance. The reagent R dropped into the well 4 has the above-described form, has therefore an increased surface area and is cooled uniformly upon cooling. The uniform cooling of the reagent R stabilizes the form of the reagent R upon anchoring. A combination of the cooling and the freeze drying prevents bumping and the like of the reagent R within the well 4 upon freeze drying, and the form of the reagent R within the well 4 can be easily kept upon dropping, even after dropping.

A part of the substance for the nucleic acid amplification reaction, which may be the reagent R anchored within the well 4 of the microchip A, may be laminated within the well 4 by repeating the dropping S3 and the anchoring S4 of the reagent R within the well 4. When the reagent R is laminated within the well 4, after the second round of the dropping S3 and the anchoring S4, in order to prevent the reagent R anchored within the well 4 from dissolving, it is desirable that the microchip A be kept at low temperature upon the dropping of the reagent R and be immediately frozen after the dropping.

(2-5) Bonding of Substrate Layers

In FIG. 4, a symbol S5 represents a bonding of the substrate layers. In S5, the substrate layer holding the reagent R within the well 4 in the anchoring S4 is bonded to other substrate layer. The bonding can be performed by using a sheet including an adhesive agent or having tackiness, thermal fusion bonding, anodic bonding, ultrasonic bonding and the like. Also, the surfaces of the substrate layer can be bonded by activating with an oxygen plasma treatment or a vacuum ultraviolet treatment. Plastic such as polydimethylsiloxane has high affinity with glass. When the surface of the substrate layer including the plastic is activated with the treatment and is contacted therewith, dangling bonds are reacted to form strong covalent bonds, i.e., Si—O—Si silanol bonds, thereby providing the adhesion having sufficient strength. Appropriate conditions of the oxygen plasma treatment or the vacuum ultraviolet light treatment are set depending on the materials of the substrate layers.

In the microchip A produced by the method of producing the microchip for the nucleic acid amplification reaction according to the embodiment of the present disclosure, the reagent R anchored within the well 4 has a certain uniform form by the hydrophilic treatment of the inner surface the well 4. When the reagent is mixed with a sample liquid fed into the microchip A, the variations between the wells 4 in degree of solubility of the reagent R anchored can be reduced. The reagent R is anchored in the form that the reagent is eccentrically-located and reduces the bulk from the marginal portion to the center portion of the well 4, thereby increasing a surface area of the reagent R. Thus, the reagent R is easily dissolved upon mixing with the sample solution. As the reagent R anchored has the above-mentioned form, it prevents the reagent R from not dissolving and remaining on the center portion of the well 4, and it can avoid detecting extremely high signal intensity on the center portion of the well 4 upon a start of the nucleic acid amplifying reaction when amplified nucleic acids are optically detected. Thus, the microchip produced by the method of producing the microchip for the nucleic acid amplification reaction according to the embodiment of the present disclosure can be used to perform the nucleic acid amplification reaction simply with high accuracy.

It should be noted that the present disclosure may also take the following configurations.

(1) A microchip for a nucleic acid amplification reaction including

a well configured to function as a reaction site of the nucleic acid amplification reaction, and the well having a center portion and a marginal portion, and

a substance anchored in a form that the substance is eccentrically-located less at the center portion and much at the marginal portion of the well, in which the substance is at least a part of a substance for the nucleic and amplification reaction.

(2) The microchip for the nucleic acid amplification reaction according to (1) above, in which

the well has a hydrophilic surface.

(3) The microchip for the nucleic acid amplification reaction according to (1) or (2) above, in which

the substance has a cone-shaped form that reduces a height from the marginal portion to the center portion of the well.

(4) The microchip for the nucleic acid amplification reaction according to any one of (1) to (3) above, in which the substance is placed circularly around the area excluding the center portion of the well.

(5) A method of producing a microchip for a nucleic acid amplification reaction including:

Applying a hydrophilic treatment to a surface of a substrate layer, on which a well configured to function as a reaction site of the nucleic acid amplification reaction is formed;

dropping at least a part of a substance for the nucleic acid amplification reaction into the well; and

drying and anchoring the substance within the well.

(6) The method of producing the microchip for the nucleic acid amplification reaction according to (5) above, in which,

the hydrophilic treatment includes exposing the surface to plasma.

(7) The method of producing the microchip for the nucleic acid amplification reaction according to (5) or (6) above, in which

the anchoring includes freeze drying the substance dropped into the well.

### EXAMPLE

Based on a method of producing a microchip according to an embodiment of the present disclosure, the microchip was produced. The form of a reagent anchored into a well and the state when the reagent was dissolved were observed.

<Materials of Microchip and Method of Producing Microchip>

On a polydimethylsiloxane (PDMS) substrate layer as a material of the microchip, a main flow channel, a branched flow channel and a well were formed. Only the surface of the PDMS substrate layer having no flow channel and no well was activated, and was bonded to a glass plate. Then, the flow channel etc. were protected by a metal mask. Only the inner surface of the well was subjected to a hydrophilic treatment by a reactive ion etching (10 cc, 50 W, 15 sec). Thus-obtained microchip as described above was used as Example. As Comparative Example, the microchip was produced as in Example except that the inner surface of the well was not subjected to the hydrophilic treatment. A reagent liquid containing a primer was dropped into each well of the microchips in Example and Comparative Example. The microchip in Example was placed at −28° C. to freeze the reagent. The microchip in Comparative Example was separated into two groups. One group was placed at −28° C. to freeze the reagent dropped as in Example. The other group was dried under reduced pressure (1000 Pa) at room temperature to anchor the reagent into the well. One group frozen at −28° C. was Comparative Example 1. The other group dried under reduced pressure was Comparative Example 2. In Example and Comparative Example 1, after the reagent was frozen, the reagent was anchored within the well in a freeze dryer (25 Pa). Then, a reagent liquid containing an enzyme was dropped and anchored into each well, into which the reagent liquid containing the primer was anchored, of the microchip in Example, Comparative Example 1 or 2. In anchoring after dropping, as in the reagent liquid containing the primer, in Example and Comparative Example 1, after frozen, the reagent was freeze dried, in Comparative Example 2, the reagent was dried under reduced pressure.

<Observation Results>

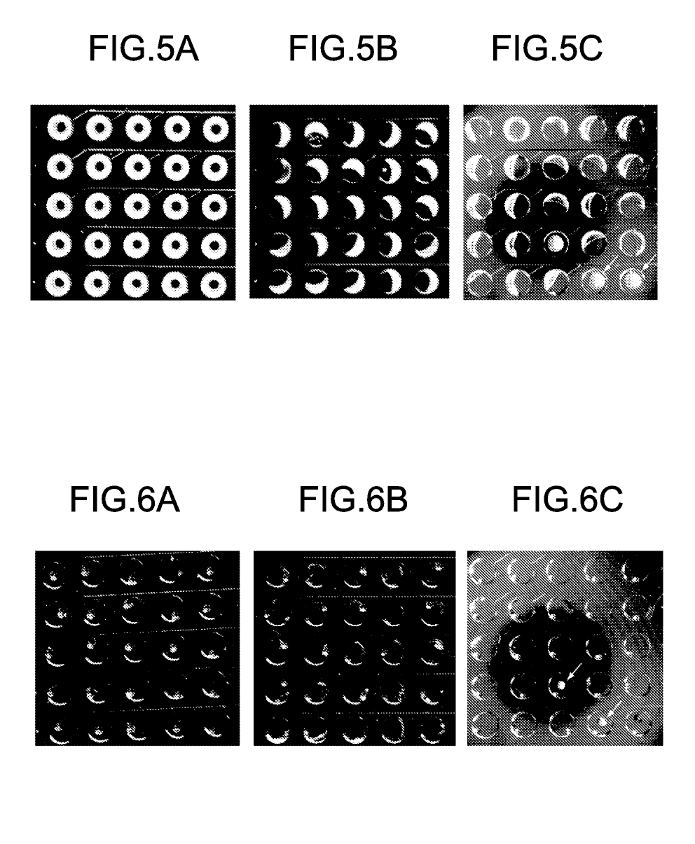

FIG. 5 show results observed from a top surface of each microchip for the form of the reagent anchored within the well in Example and Comparative Examples 1 and 2. As shown in FIG. 5A, in Example, the reagent was anchored circularly excluding the center portion of each well. The form of the reagent anchored was uniform between the wells. As shown in FIG. 5B, in Comparative Example 1, the reagent anchored within each well was concentrated at one side of the inner surface of each well, and was non-uniform. As shown in FIG. 5C, in Comparative Example 2, the reagent was eccentrically anchored at one side of the inner surface of each well as in Comparative Example 1, but in some wells the reagent was concentrated at the center portion thereof (shown by arrows in FIG. 5C).

A buffer solution for the nucleic acid amplification reaction was added to each well in Example, and Comparative Examples 1 and 2, and the state of the reagent within each well was observed after 30 seconds. FIG. 6 show the wells observed from a top surface of each microchip. FIG. 6A shows the wells in Example. FIGS. 6B and 6C show the wells in Comparative Examples 1 and 2, respectively. Although, in any one of Example and Comparative Examples 1 and 2, it was observed that the reagent anchored was not dissolved and remained, in Comparative Example 2, it was observed that a large amount of the reagent was not dissolved and remained the center portion of the wells (shown by arrows in FIG. 6C).

The results in Example reveal that the hydrophilic treatment of the inner surface of the well and the sufficient freezing of the reagent before drying affect the form of the reagent anchored within the well of the microchip. Also, it is revealed that the reagent is differently dissolved and remained depending on the form of the reagent anchored.

By the microchip for the nucleic acid amplification reaction according to the embodiment of the present disclosure, the analysis can be performed simply with high accuracy. Thus, the microchip for the nucleic acid amplification reaction according to the embodiment of the present disclosure can be used as a nucleic acid amplification apparatus for clinical genotyping, contagium determination and the like.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A microchip for a nucleic acid amplification reaction, comprising:
a well configured to function as a reaction site of the nucleic acid amplification reaction, and the well having a center portion and a marginal portion, and
a substance anchored in a form that the substance is eccentrically-located less at the center portion and much at the marginal portion of the well, wherein the substance is at least a part of a substance for the nucleic and amplification reaction.

2. The microchip for the nucleic acid amplification reaction according to claim 1, wherein
the well has a hydrophilic surface.

3. The microchip for the nucleic acid amplification reaction according to claim 2, wherein
the substance has a cone-shaped form that reduces a height from the marginal portion to the center portion of the well.

4. The microchip for the nucleic acid amplification reaction according to claim 3, wherein
the substance is placed circularly around the area excluding the center portion of the well.

5. A method of producing a microchip for a nucleic acid amplification reaction comprising:
applying a hydrophilic treatment to a surface of a substrate layer, on which a well configured to function as a reaction site of the nucleic acid amplification reaction is formed;
dropping at least a part of a substance for the nucleic acid amplification reaction into the well; and
drying and anchoring the substance within the well.

6. The method of producing the microchip for the nucleic acid amplification reaction according to claim 5, wherein,
the hydrophilic treatment includes exposing the surface to plasma.

7. The method of producing the microchip for the nucleic acid amplification reaction according to claim 6, wherein
the anchoring includes freeze drying the substance dropped into the well.
